(19) United States
(12) Patent Application Publication
(10) Pub. No.: US 2012/0116422 A1
(43) Pub. Date: May 10, 2012

(54) SUTURE PASSERS

(75) Inventors: Daniel J. Triplett, Providence, UT (US); Carlyle J. Creger, Wellsville, UT (US)

(73) Assignee: MEDICINELODGE, INC. DBA IMDS CO-INNOVATION, Logan, UT (US)

(21) Appl. No.: 13/293,856

(22) Filed: Nov. 10, 2011

Related U.S. Application Data
(60) Provisional application No. 61/412,228, filed on Nov. 10, 2010.
(60) Provisional application No. 61/441,169, filed on Feb. 9, 2011.
(60) Provisional application No. 61/441,185, filed on Feb. 9, 2011.

(51) Int. Cl. A61B 17/04; B23P 11/00

(52) U.S. Cl. 606/144; 29/428

(57) ABSTRACT

Suture passers which include a handle assembly, shaft assembly, and jaw assembly. In one embodiment, the handle assembly includes first and second handles joined at a hinge, the second handle pivotable relative to the first handle and guided by an arcuate guiding feature concentric to the hinge. The jaw assembly includes upper and lower jaws, the lower jaw including an open needle track. The open needle track includes entry, tangential and exit segments, each of which is directly visible, directly accessible, and directly inspectable. The lower jaw can be manufactured as a single piece, using a single manufacturing process, which may be injection molding or multi-axis machining. When manufactured by multi-axis machining, all features of the lower jaw may be machined without removal of the jaw piece from the multi-axis machine. Methods of suture passer manufacture and assembly which may provide significant cost savings are disclosed.

### CROSS-REFERENCE TO RELATED APPLICATIONS

This application is a non-provisional of:

pending U.S. Provisional Patent Application No. 61/412,228, filed Nov. 10, 2010, which carries Applicant's docket No. MLI-91 PROV, and is entitled SUTURE PASSER; and

pending U.S. Provisional Patent Application No. 61/441,169, filed Feb. 9, 2011, which carries Applicant's docket No. MLI-95 PROV, and is entitled SUTURE PASSER; and

pending U.S. Provisional Patent Application No. 61/441,185, filed Feb. 9, 2011, which carries Applicant's docket No. MLI-96 PROV, and is entitled SUTURE PASSER.

The above-identified documents are incorporated herein by reference.

### FIELD OF THE DISCLOSURE

This disclosure relates to surgical suturing devices by which suture may be passed through tissue during surgery and methods of manufacture for surgical suturing devices. Specifically, this disclosure relates to methods of manufacture and assembly for a low-cost, disposable suturing device.

### BACKGROUND OF THE INVENTION

Reusable instruments are costly; therefore many hospitals limit the purchase of these instruments to a reasonable minimum. This can correspondingly limit the number of associated medical procedures that the surgical staff can complete in a given day. The number of procedures performed in a day may be governed by how fast the hospital's central processing can turnaround an instrument through re-use processes which may include scheduling, cleaning, sterilization, re-shelving and inventory procedures. An alternative to reusable instruments and the re-use processes associated with them is to provide a supply of disposable instruments which are capable of performing the same medical procedures, but which do not require the scheduling, cleaning, sterilization and re-shelving procedures. Additionally, purchase of expensive, reusable instruments is commonly accompanied by a product warranty and/or a maintenance program. Moving to a disposable instrument supply eliminates the tasks associated with managing service programs and warranties. Another factor in surgical instrument overall cost and use is the degradation of instrument performance over time. For example, over time and repeated use, a suture passer may require increased needle deployment forces, and needle life may be reduced. A disposable instrument will result in a more consistent performance for the surgeon.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is an isometric side view of a disposable suture passer including a handle assembly, a shaft assembly, and a jaw assembly;

FIG. 2A is a distal isometric view of a fixed handle of the suture passer of FIG. 1;

FIG. 2B is a proximal isometric view of the fixed handle of FIG. 2A;

FIG. 3A is a superior view of the fixed handle of FIG. 2A; FIG. 3B is a cross-sectional side view of the fixed handle of FIG. 3A taken along line A-A of FIG. 3A;

FIG. 4A is a side view of a moving handle of the suture passer of FIG. 1; FIG. 4B is a distal isometric view of the moving handle of FIG. 4A;

FIG. 5A is a superior view of the moving handle of FIG. 4A; FIG. 5B is a cross-sectional side view of the fixed handle of FIG. 5A taken along line B-B of FIG. 5A;

FIG. 6 is an enlarged isometric view of a hinge pin of the suture passer of FIG. 1;

FIG. 7 is an enlarged isometric view of a trigger of the suture passer of FIG. 1;

FIG. 8A is an enlarged isometric view of a trigger anvil of the suture passer of FIG. 1; FIG. 8B is an isometric cross-sectional view of the trigger anvil of FIG. 8A, taken along line C-C in FIG. 8A;

FIG. 9 is an enlarged isometric side view of a plug of the suture passer of FIG. 1;

FIG. 10 is an exploded view of the shaft assembly of FIG. 1, including an outer sleeve, a trigger pushrod, and a needle;

FIG. 11 is an exploded view of the jaw assembly of FIG. 1, including an upper jaw, a lower jaw, a jaw link, connecting pins, and the distal end of the trigger pushrod;

FIG. 12A is a side view of the lower jaw of FIG. 11; FIG. 12B is a superior view of the lower jaw of FIG. 12A; FIG. 12C is a side cross-sectional view of the lower jaw of FIG. 12B, taken along line D-D in FIG. 12B, a dashed line representing the path of a needle through the lower jaw;

FIG. 13 is a side isometric view of the jaw assembly of FIG. 11 and outer sleeve and pushrod of FIG. 10, assembled together to form an assembly;

FIG. 14 is a side isometric view of the assembly of FIG. 13, with the addition of the plug and trigger anvil, and a spring;

FIG. 15 is a side isometric view of the assembly of FIG. 14, with the addition of the fixed handle;

FIG. 16 is a side isometric view of the assembly of FIG. 15, with the addition of the trigger;

FIG. 17 is a side isometric view of the assembly of FIG. 16, with the addition of the needle, a main spring, and a trigger pocket cap;

FIG. 18A is a side cross-sectional view of the suture passer of FIG. 1 taken through a plane of bilateral symmetry of the suture passer, with the jaw assembly in an open configuration; FIG. 18B is a side cross-sectional view of the suture passer of FIG. 1, with the jaws in a closed configuration and the needle in an extended position;

FIG. 19A is a superior view of a locking member; FIG. 19B is an isometric view of the locking member of FIG. 19A;

FIG. 20 is an isometric view of the suture passer of FIG. 1 with the locking member of FIG. 19A, the trigger pocket cap removed to show the interaction of the locking member with the trigger anvil;

FIG. 21 is a side view of an alternative embodiment of a disposable suture passer, including a handle assembly, a shaft assembly, and a jaw assembly;

FIG. 22 is a side cross-sectional view of the handle assembly of the suture passer of FIG. 21 taken through a plane of bilateral symmetry of the suture passer;

FIG. 23 is a superior isometric view of a trigger assembly of the suture passer of FIG. 21, a fixed handle removed to show the detail of the trigger assembly;

FIG. 24 is a superior isometric view of the jaw assembly of the suture passer of FIG. 21, the jaws in an open configuration; and

FIG. 25 is a side view of another alternative embodiment of a disposable suture passer.

### DETAILED DESCRIPTION OF THE PREFERRED EMBODIMENTS

The present disclosure relates to suture passing devices and methods of manufacture of these devices. Those of skill in the art will recognize that the following description is merely illustrative of the principles of the disclosure, which may be applied in various ways to provide many different alternative embodiments. This description is made for the purpose of illustrating the general principles of this invention and is not meant to limit the inventive concepts in the appended claims.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

In the context of this application, a re-usable instrument means an instrument intended for use in more than one discrete surgical procedure. Re-use of an instrument may require cleaning, sterilization, inspection, inventory, and/or scheduling processes between uses. In the context of this application, a disposable instrument means an instrument intended for use in only one discrete surgical procedure, after which the instrument is discarded. It is understood that a discrete surgical procedure may involve one or more instrument actuations.

In the context of this application, a single manufacturing process is a manufacturing process carried out on one machine using one fabrication method and one setup of the machine to form substantially all of the features, geometry, and/or dimensions of a net shape component in its final usable condition. For example, a single process may be injection molding in which a single injection, shot, or bolus of moldable material is introduced into a single mold body. Another single manufacturing process may be multi-axis machining, wherein a coupon or workpiece is mounted on the machine, and in a single setup of the machine and without removal of the workpiece, the various features of the component are created in their entirety. Yet another single manufacturing process may be additive manufacturing, the process of joining materials to make objects from 3D model data, usually layer upon layer, as opposed to subtractive manufacturing methodologies, such as traditional machining. A single manufacturing process may also be described as a discrete manufacturing process, in the sense that it is individually separate and distinct from other manufacturing processes. The net shape component may undergo subsequent finishing, polishing, plating, passivating, anodizing, coating, etching, reaming, thread chasing, and/or related steps which have minimal or negligible effect on the component's features, geometry, and/or dimensions.

In the context of this application, a single piece is a unitary piece which does not have any other components permanently added to it to be complete in its final, usable form. For example, the lower jaw piece with needle track described herein is manufactured as a single piece: no additional parts such as a needle track cover need to be added to it for it to be complete and usable. A single piece may also be described as a discrete piece.

According to a first aspect of the invention, a method of manufacturing a suture passer having a jaw component having a nonlinear track formed therethrough includes manufacturing the jaw component as a single piece, wherein manufacturing the jaw component consists essentially of forming substantially all of the geometry and dimensions of the jaw component in a single manufacturing process.

Embodiments of this aspect of the invention may include one or more of the following features. The track enters the jaw component at a first opening and exits jaw component at an exit opening offset from the first opening. The track includes an entry segment, a tangential segment, and an exit segment. The tangential segment is in communication with the entry segment and the exit segment, forming a continuous path through the jaw component. Every segment of the track is directly accessible and directly visible from outside the jaw component

In an embodiment, the method further includes confirming patency of the track through direct inspection.

In an embodiment, the single manufacturing process is a single molding process. Embodiments of the single molding process may include one or more of the following features or methods. Injecting a single bolus of material into a single mold body. Inserting at least one core into the mold body to create an internal feature in the jaw component. The jaw component includes at least one material chosen from the group consisting of: metal, ceramic, plastic, and carbon fiber. The single molding process is a process selected from the group consisting of centrifugal casting, die casting, and metal injection molding.

In an embodiment, the single manufacturing process is multi-axis machining. Embodiments of multi-axis machining may include one or more of the following features or methods. Manufacturing the jaw component using a single setup of a multi-axis machining center. Mounting a workpiece on the multi-axis machining center. Forming substantially all of the geometry and dimensions of the jaw component on the workpiece without removing the workpiece from the multi-axis machining center. Forming substantially all of the geometry and dimensions of the jaw component by forming a first segment of the track into the workpiece from a first direction; forming a second segment of the track into the workpiece, the second segment offset from the first segment; and forming a third segment of the track into the workpiece from a second direction, the second direction oriented at an angle relative to the first direction. The angle ranges from 45° to 135° relative to the first direction.

According to a second aspect of the invention, a method of manufacturing a suture passer, the suture passer having a jaw component having a nonlinear track formed therethrough includes the step of forming substantially all of the jaw component in a single manufacturing process.

Embodiments of this aspect of the invention may include one or more of the following features or steps. The track enters the jaw component at a first opening and exits the jaw component at an exit opening offset from the first opening. The track includes an entry segment, a tangential segment, and an exit segment. The tangential segment is in communication with the entry segment and the exit segment, forming a continuous path through the jaw component. Every segment of the track is directly accessible and directly visible from outside the jaw component. The step of confirming patency of the track though direct inspection.

In an embodiment, the single manufacturing process is a single molding process. Embodiments of the single molding process may include one or more of the following features or steps. The step of introducing a single bolus of material into a single mold body. The step of inserting at least one core into the mold body to create an internal feature in the jaw component. The jaw component comprises at least one material chosen from the group consisting of: metal, ceramic, plastic, and carbon fiber. The single molding process is a process selected from the group consisting of centrifugal casting, die casting, and metal injection molding.

In an embodiment, the single manufacturing process is multi-axis machining. Embodiments of the multi-axis machining process may include one or more of the following features or steps. The step of manufacturing the jaw component using a single setup of a multi-axis machining center. The step of mounting a workpiece on the multi-axis machining center. The step of forming substantially all of the geometry and dimensions of the jaw component on the workpiece without removing the workpiece from the multi-axis machining center. The step of forming a first segment of the track into the workpiece from a first direction. The step of forming a second segment of the track into the workpiece, the second segment offset from the first segment. The step of forming a third segment of the track into the workpiece from a second direction, the second direction oriented at an angle relative to the first direction. The angle ranges from 45° to 135° relative to the first direction.

According to a third aspect of the invention, a handle assembly for a surgical instrument includes a first handle and a second handle, the first and second handles pivotably joined by a hinge; the first handle further comprising an arcuate guide offset from the hinge, the arcuate guide concentric to the hinge; the second handle further comprising a guided feature; the arcuate guide cooperates with the guided feature to guide pivotal movement of the second handle relative to the first handle about the hinge.

Embodiments of this aspect of the invention may include one or more of the following features. The arcuate guide comprises an arcuate slot. The guided feature includes a pin which travels in the arcuate slot to constrain the pivotal movement of the second handle relative to the first handle. The guided feature includes a clevis formed on the second handle, and the clevis carries the pin. A spring captured between the first handle and the second handle, the spring biasing the handle assembly toward an open configuration. The spring lies between the hinge and the arcuate guide.

The handle assembly includes an instrument shaft having a shaft axis. A proximal end of the instrument shaft is seated in the handle assembly. The pivotal movement of the second handle relative to the first handle translates the instrument shaft along the shaft axis. The second handle includes a shaft seat, and the proximal end of the instrument shaft is seated in the shaft seat. The instrument shaft lies between the hinge and the arcuate guide. The shaft axis, hinge, guide feature and arcuate guide all lie in the same plane of symmetry.

The handle assembly includes a first ring and a second ring, the first and second rings fitted concentrically together, the first and second rings pivotably movable relative to one another. The first handle comprises the first ring and the second handle comprises the second ring. The first ring comprises a tab and the second ring comprises a slot. The tab is received in the slot to lock the first and second rings concentrically together.

The first and second handles are formed by an injection molding process.

The guided feature, arcuate guide, and hinge are all in the same plane of symmetry.

The suture passer embodiments described herein may be manufactured employing procedures which may significantly reduce the cost of manufacture compared to the cost of manufacture of a re-usable suture passer. Some of these cost-saving procedures or methods may include: injection molding of plastic handle pieces; inclusion of a single lower jaw piece which may be manufactured using a single manufacturing process in a single setup; and inclusion of a needle track which is exposed, allowing for direct visual inspection. Other cost saving manufacturing methods may be employed and these may be noted throughout the disclosure. It is appreciated that although methods of manufacture for a disposable suture passer are set forth, some or all of the methods may be applied to manufacture of a re-usable suture passer.

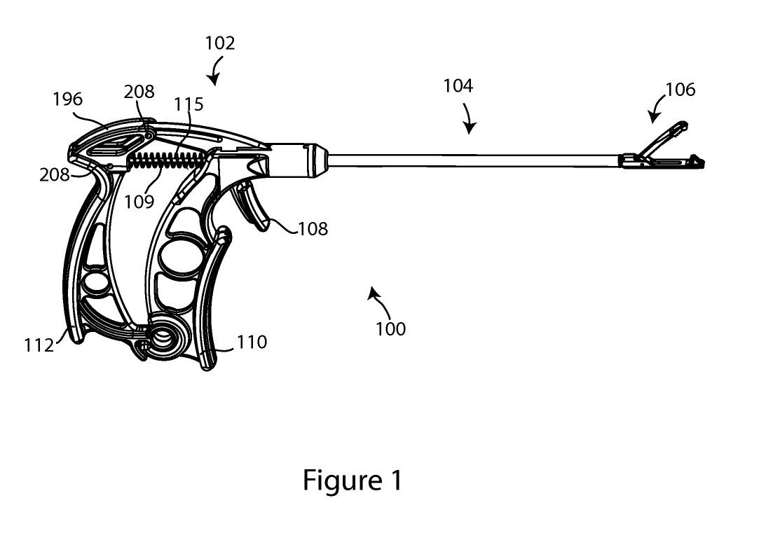

Referring to FIG. 1, a disposable suture passer 100 according to one embodiment of the invention is shown. Suture passer 100 includes a handle assembly 102, a shaft assembly 104, and a working end 106. A suture needle 109 is anchored in the handle assembly, passes through the shaft assembly, and a needle tip capable of carrying a suture forms part of the working end 106. Other needle tip styles may be adapted for other functions, such as piercing tissue, instead of carrying a suture. The working end includes opposed jaws and the needle distal end and tip, and may be referred to as a jaw assembly. The handle assembly 102 includes a trigger 108 actuable to open and close the jaws, and the handle assembly may be actuated to deploy the needle to penetrate and carry the suture through targeted tissue, and retract the needle. These steps may be repeated numerous times during a single surgical procedure. One example of a suture passer embodying some of the elements disclosed herein is shown in U.S. Design patent application Ser. No. 29/383,019, which application is hereby incorporated by reference in its entirety.

Handle assembly 102 further includes a fixed handle 110 which may be a first handle, and moving handle 112 which may be a second handle. The fixed handle 110 and moving handle 112 may be joined at least two points, and the moving handle 112 may pivot and/or translate relative to the fixed handle 110 to actuate the needle. A main spring 115 may be positioned between the fixed and moving handles 110, 112. The main spring 115 may bias the handles 110, 112 apart and bias the needle toward a retracted position; by squeezing the handles together an operator may overcome the spring bias and advance the needle into an extended position. The handles 110, 112 move between an open position in which they are spaced apart and a closed position in which they are relatively closer together. It is appreciated that in another embodiment, the positions of the fixed and moving handles may be reversed. In another embodiment, both handles may move relative to one another or other portions of the suture passer. In yet another embodiment, the handle may comprise one piece having a moving portion. The fixed and moving handles 110, 112, may be formed using traditional plastic injection molding processes and may be formed from plastics such as acrylonitrile butadiene styrene (ABS), polycarbonate, or a blend.

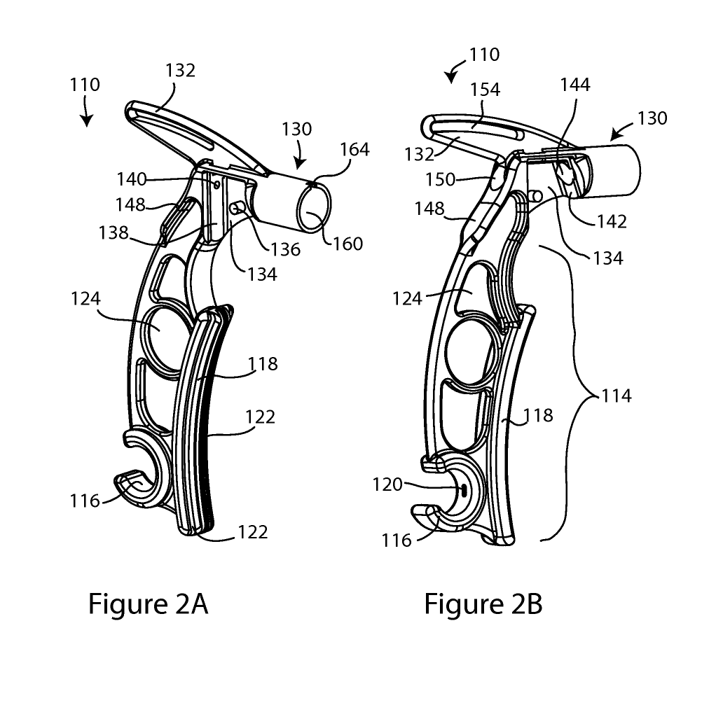

Referring to FIGS. 2A-B and 3A-B, fixed handle 110 includes a body portion 114 which further includes a first or outer hinge section 116, and a first grip 118. First hinge section 116 is ring-shaped to concentrically fit with a hinge section on the moving handle. This arrangement does not require a separate pin component to complete the hinge. The hinge section 116 may include a tab 120 at its center plane that snaps into a slot centered in the moving handle to lock the components into a proper alignment between the two components. The first grip 118 may have a length, width and curvature designed to be comfortable in a surgeon's hand. A plurality of longitudinally aligned grooves 122 may be formed on the first grip 118 to reduce the amount of material needed to form the grip, while still maintaining sufficient rigidity for proper functioning of the handle assembly. One or more openings 124 may be formed in the body portion 114. Their presence may reduce material needed, reduce weight and cost, and/or provide aesthetic appeal while still providing proper handle functionality.

The fixed handle 110 further includes a barrel portion 130 which is shaped to receive the needle and the proximal end of the shaft assembly. A guide portion 132 is formed to cooperate with the moving handle to guide movement of the moving handle relative to the fixed handle. A trigger pocket 134 is formed proximal to the barrel portion, and includes a first boss 136 which functions as a trigger pin to allow pivoting of the trigger 108. A proximal wall 138 of the trigger pocket 134 includes a needle aperture 140 to allow passage of a shaft portion of the needle 109. A distal wall 142 of the trigger pocket 134 includes an anvil pocket 144. A needle passage 146 is formed from anvil pocket 144 and opens into the barrel portion 130, and several steps may be formed into the sidewall of the needle passage 146. A transition portion 148 is formed to provide a transitional structure between the body portion 114, the trigger pocket 134 and the guide portion 132. The transition portion 142 includes a first spring pocket 150 which communicates with the first needle aperture 140; the first needle aperture 140 may be positioned in the center of the first spring pocket 150. The first needle aperture 140 is coaxially aligned with needle passage 146, and may serve to constrain the needle along a fixed trajectory.

The guide portion 132 includes a range of motion track 154, which may be described as a guide. The range of motion track 154 may be shaped as an arcuate slot, and is located on the top of the fixed handle 110 in the main plane of symmetry. The range of motion track 154 is concentrically aligned with the first hinge section 116. The range of motion track 154 provides a range of motion stop for the moving handle 112 as the main spring 115 acts to retract the needle. It also provides lateral support for the moving handle. Both of these functions allow for a lighter hinge design and structure between the hinge and needle region of the instrument. The range of motion track 154 may be another negative feature, such as a groove; it may also be a positive feature, such as a rail, in other examples.

The barrel portion 130 may be cylindrical, and includes a plug receptacle 160. The plug receptacle 160 allows for the shaft assembly 104 to be assembled separately and then added to the instrument in a single assembly step. The receptacle 160 includes an end wall 162 which functions as a stop for the shaft assembly, and a notch 164 which functions as a keying feature to ensure that the shaft assembly 104 is properly clocked, or oriented, to the handle assembly Other keying features are within the scope of the invention, including but not limited to slots, grooves, threads, snap features, and complementarily shaped surfaces such as a hex connection or other complementarily shaped connections.

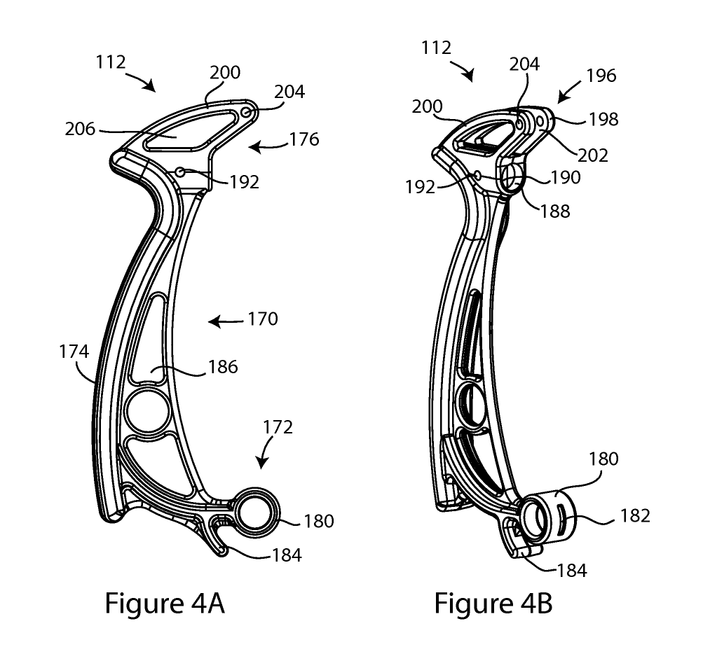

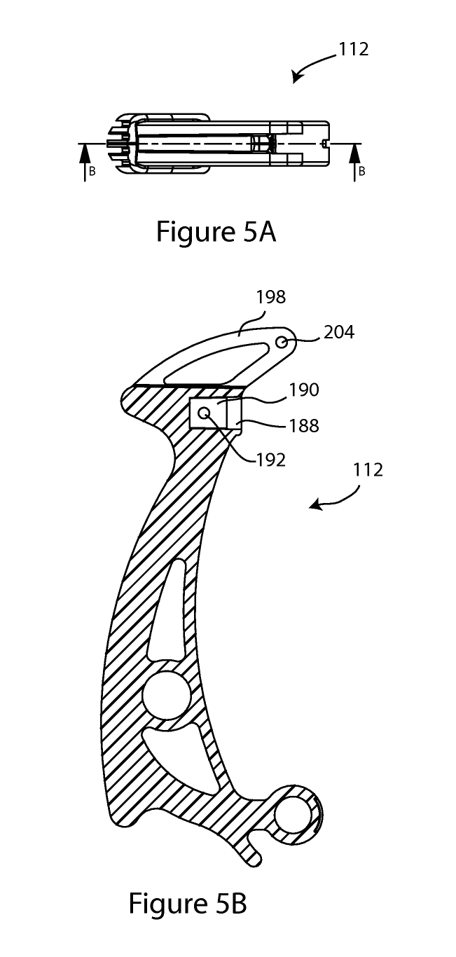

Referring to FIGS. 4A-B and 5A-B, the moving handle 112 includes a handle body 170, second or inner hinge section 172, second grip 174, and connection portion 176. Second hinge section 172 includes a ring 180 shaped to concentrically fit within hinge section 116 on the fixed handle 110, to form a hinge which allows the moving handle 112 to pivot relative to fixed handle 110 about an axis defined by the ring 180. The ring 180 has a slot 182 at its center plane that receives tab 120 on fixed handle 110 to maintain the proper alignment between the two components in the plane of symmetry. A guard 184 may function as a pinch protection feature to ensure that the surgeon's gloves or other objects do not get caught in the hinge as it moves. The guard 184 may also cooperate with hinge section 116 to function as a stop feature to limit rotation of moving handle 112 relative to fixed handle 110. The second grip 174 may have a length, width and curvature designed to be comfortable in a surgeon's hand, and may include longitudinal grooves similar to those described for fixed handle 110. One or more openings 186 may be formed in the body portion 170.

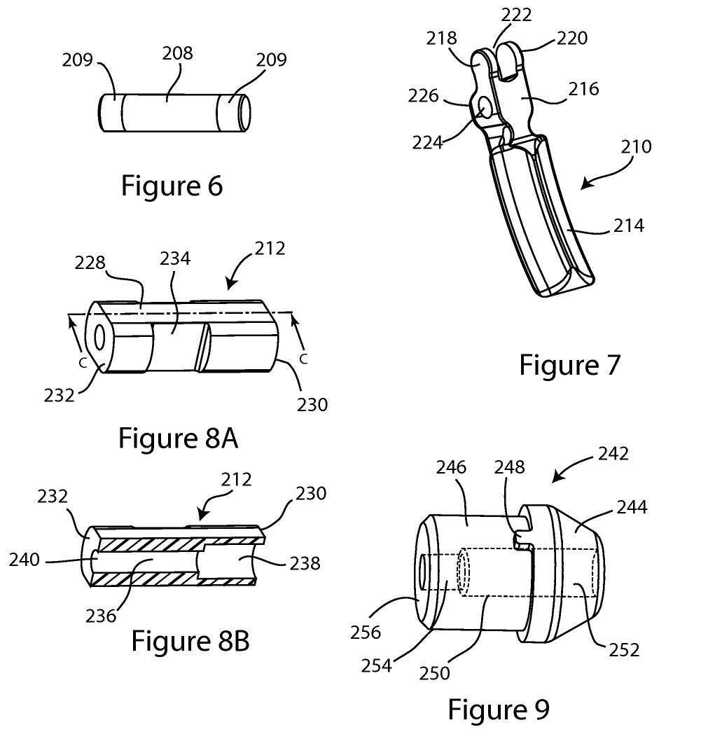

The connection portion 176 includes a second spring pocket 188. Immediately proximal to, concentric with, and in communication with the spring pocket 188 is a needle slot 190. The needle slot 190, which may be described as vertically oriented, provides a specific orientation to the needle when the needle is assembled in the instrument, and serves as a seat for a shaft of the needle. A first pair of coaxially aligned pin holes 192 extend from the needle slot 190 to the outside of the moving handle 112. The connection portion includes a clevis 196 comprising a first arm 198 positioned opposite a second arm 200, and a gap 202 formed between the arms 198, 200. The gap 202 is sized and shaped to receive the guide portion 132 of fixed handle 110 between the arms. The clevis 196 occupies the same plane of symmetry as the hinge ring 180 and hinge section 116. A second pair of coaxially aligned pin holes 204 is formed through the arms 198, 200. Each arm 198, 200 may include one or more openings 206. Referring to FIG. 6, a range of motion pin, or hinge pin 208 is sized to fit into pin holes 204. Hinge pin 208 may include one or more knurled regions 209, as the knurled surface may form a secure connection with the plastic surfaces of the pin holes 204. The hinge pin 208 and other pins in the instrument may be manufactured from standard stainless steel rod stock on a Swiss turning machine or similar high volume manufacturing process.

Moving handle 112 may be assembled with fixed handle 110 as in FIG. 1, with guide portion 132 received between arms 198, 200 and a hinge pin 208 extending through pin holes 204 and track 154. Ring 180 is received in hinge section 116 to form a hinge, allowing pivoting of moving handle 112 relative to fixed handle 110. The pin 208 and track 154 form a guide and mechanical stop to the pivoting hinge action. The arms 198, 200 on either side of guide portion 132 add strength and stability to the handle, while openings 124, 186 and 206 decrease weight. In other examples, the pin 208 may be replaced with a negative feature, such as a socket, hole, or aperture, into which a protruding track 154 may fit.

Referring to FIGS. 1, 2A, 2B, 7, 8A, and 8B, a trigger 210 and trigger anvil 212 are sized to fit into trigger pocket 134 of the fixed handle 110; a portion of trigger 210 extends out of the pocket. The trigger 210 may also be referred to as a jaw trigger or jaw actuator, as the actuation of the trigger 210 opens and closes the jaw members of the working end 106. The trigger 210 includes a pull member 214 and pivot portion 216. The pivot portion 216 includes first and second ears 218, 220, between which a gap 222 is formed. A bore 224 extends transversely through the pivot portion 216, and is sized to receive boss 136 as trigger 210 is assembled with fixed handle 110. A curved cam surface 226 is formed on the pivot portion 216. The trigger anvil 212 is sized to fit between the proximal 138 and distal 142 walls of the trigger pocket 134, seen in FIGS. 2A and 2B. The trigger anvil 212 includes an anvil body 228 extending between a first end 230 and a second end 232. A waist 234 is situated partway between the first and second ends where the width of the anvil body 228 is reduced. The waist 234 is sized to fit in the gap 222 of the trigger 210, between the ears 218, 220. The rounded shape of the ears allows pivoting of the trigger 210 relative to the anvil 212 without need for a discrete joint pin. An anvil bore 236 extends lengthwise through the anvil body 228, and may include a first bore segment 238 which is sized to receive and seat one end of a jaw pushrod, and a second bore segment 240 which is sized to allow passage of a shaft of needle 109. The trigger anvil 212 may also be referred to as an actuator, as the actuation of the trigger anvil 212 opens and closes the jaw members of the working end 106. Together the trigger 108 and anvil 212 may be referred to as a trigger assembly.

Referring to FIG. 9, a plug 242 is shaped to fit into plug receptacle 160, seen in FIG. 2A on fixed handle 110. Plug 242 includes a plug cap 244 and a plug body 246 which 4 E u may be of smaller diameter than the plug cap. A key 248 is shaped to cooperate with notch 164, to provide proper indexing between the components. A stepped plug bore 250 extends internally along the length of the plug 242. A first plug bore segment 252 is sized to receive one end of an outer sleeve and ensure its insertion to a proper depth. A second plug bore segment 254 is sized to allow passage of a pushrod and the shaft of needle 109. The second plug bore segment 254 opens out at a proximal end face 256 of the plug. The trigger 210, trigger anvil 212, and plug 242 may be formed using traditional plastic injection molding processes and may be formed from plastics such as ABS, polycarbonate, or a blend.

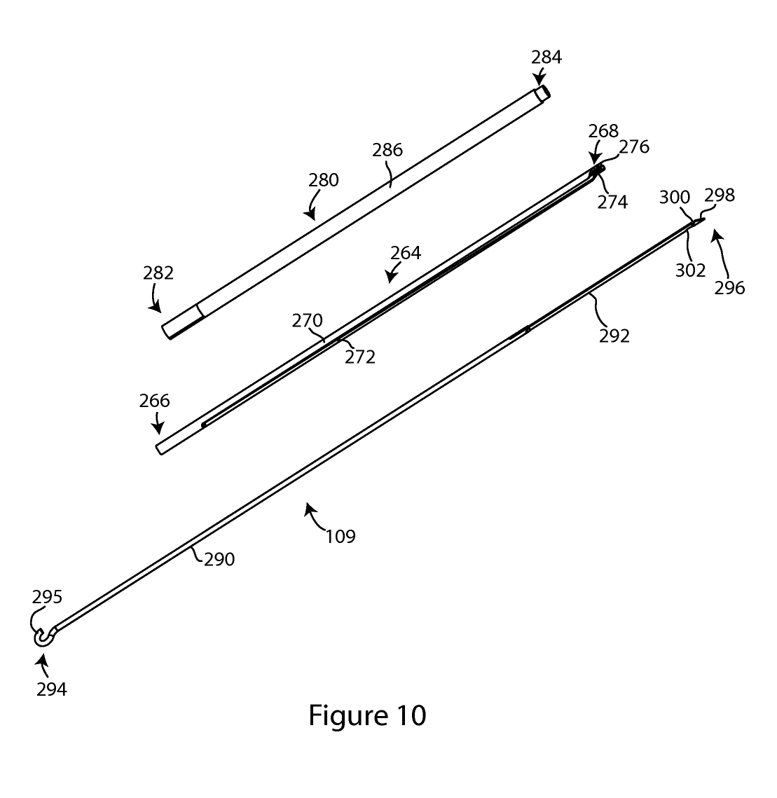

FIG. 10 is an exploded view of components included in the shaft assembly 104. Pushrod 264 includes a first end 266 which may be a proximal end, a second end 268 which may be a distal end, and a pushrod shaft 270 extending therebetween. The first end 266 is sized to be concentrically received in first bore segment 238 of the anvil 212. A groove 272 extends along a portion of the length of the shaft 270, and opens out at the second end 268. A pushrod clevis 274 projects from the second end 268, and includes a pair of holes 276 for connection with working end 106. Outer sleeve 280 is a generally tubular member which includes a first end 282 which may be a proximal end, a second end 284 which may be a distal end, and a sleeve shaft 286 extending therebetween. First end 282 may be knurled to promote tight connection within plug bore segment 252. Second end 284 may be stepped, having a reduced diameter to facilitate connection with a jaw member. Pushrod 264 and outer sleeve 280 may be manufactured from stainless steel on a Swiss turning machine or similar high volume manufacturing process.

Needle 109 includes an elongated, generally cylindrical needle shaft 290, joined to an elongated, generally flattened needle ribbon 292. A proximal or first end 294 of the needle 109 forms a hook, or eyelet 295, to facilitate connection to moving handle 112. A distal or second end 296 of the needle 109 includes a needle tip 298 and an eye 300 to facilitate carrying of suture. A concavity 302 may be formed on the opposite side of the needle from the eye 300. The rounded needle shaft 290 may be more rigid than the flattened ribbon 292, and may provide needle deployment force through the instrument shaft. The needle shaft 290 may be constructed from a superelastic alloy such as Nitinol or stainless steel, among other materials. The needle ribbon 292 may be constructed from Nitinol; wire EDM (electronic discharge machining) may be used to provide the tip features including the eye 300 and the concavity 302. The needle shaft 290 and ribbon 292 may be welded together.

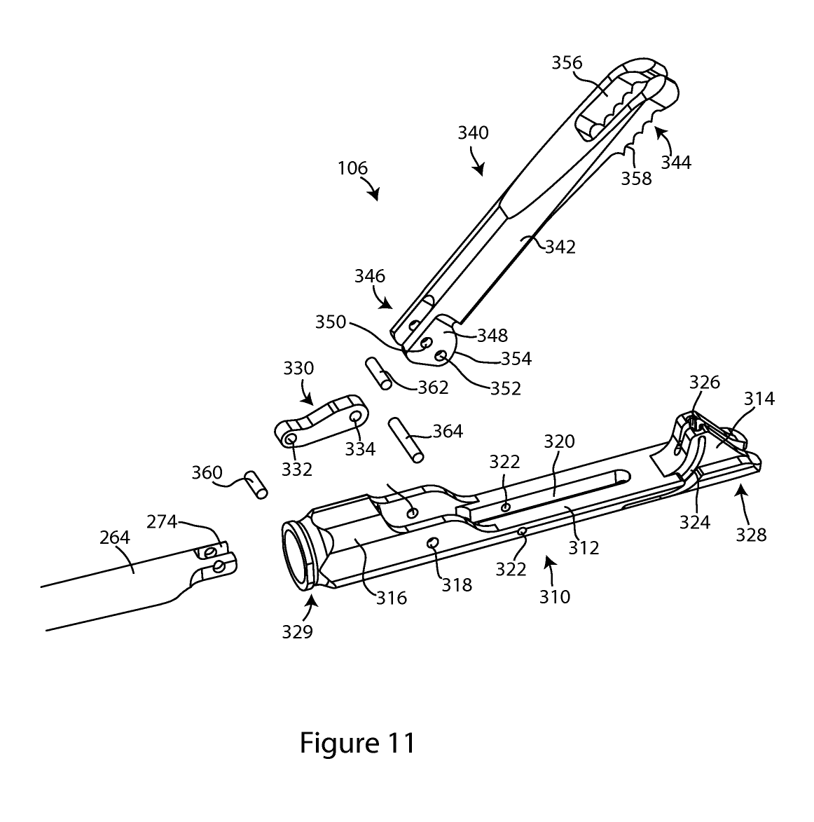

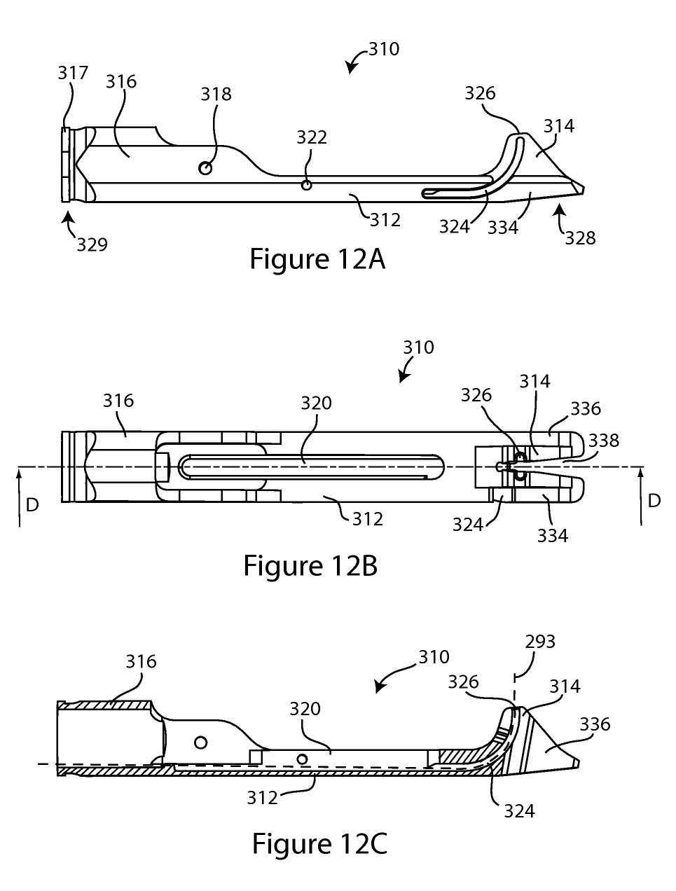

Referring to FIG. 11, an exploded view of the working end 106 includes a distal portion of the pushrod 264, a lower jaw 310, a jaw link 330, and an upper jaw 340. More details of the lower jaw 310 are seen in FIGS. 12A-12C. The lower jaw 310 includes a relatively flat jaw plate 312 which extends between the proximal and distal ends of the lower jaw. Toward a distal end 328 of the lower jaw 310, a needle guide 314 projects superiorly from the jaw plate 312. Toward a proximal end 329 of the lower jaw 310, a link housing 316 is formed, which houses the distal end of the pushrod 264 and the jaw link 330, and provides a site for attachment of the upper jaw 340. At the proximal end 329, a flange 317 may be provided, and may supply melt material for joining the lower jaw 310 to the outer sleeve 280. A set of upper jaw pin holes 318 are formed in the link housing 316 to provide linkage to the upper jaw. A jaw pocket 320 is formed as an elongated groove or recess in the jaw plate 312, and a set of track pin holes 322 may be formed on either side of the jaw pocket 320. A curved jaw track 324 is formed in communication with the jaw pocket 320 at one end, and opens out at a jaw track opening 326 on the needle guide 314. When the needle 109 is extended the needle ribbon 292 extends longitudinally through the jaw pocket 320, is redirected tangentially by the curved jaw track 324, and the needle distal end 296 exits out of the jaw track opening 326. Jaw pocket 320, curved jaw track 324 and track opening 326 may be said to form a needle track, in which the jaw pocket forms a first segment of the track, the curved jaw track forms a second or tangential segment, and the track opening forms a third or exit segment. The needle track may be described as a continuous path through the jaw component. The link housing 316 and jaw pocket 320 also form an entry segment of the track. In FIG. 12C, dashed line 293 represents the needle track through the lower jaw 310. The needle track may be nonlinear, as shown. The jaw distal end 330 may be forked, forming a first prong 334 and a second prong 336 separated by a suture gap 338. The suture gap 338 is in communication with the curved jaw track 324 and jaw track opening 326.

The lower jaw 310 may be formed by a metal injection molding process as a single component. The jaw component may then be machined on a single multi-axis mill using a single setup to form the jaw pocket 320, curved needle track 324, and track opening 326. After the machining operation, the lower jaw 310 may be a single net shape component having substantially all of its final features, geometry, and/or dimensions. The use of a single machine and a single setup may provide significant cost savings, as the component can be made with minimal operator interaction during the manufacturing process. This single machine, single setup process may be viewed in comparison to traditional methods for manufacture an assembly of a suture passer jaw, which methods often include multiple steps of machine fabrication with multiple setups, first inspection, welding of components such as a needle track cover, additional inspection, wire EDM cutting of a suture notch, patency check with a flexible gauge, and a final inspection. In contrast, the mill operations to cut the jaw pocket 320, curved needle track 324, and track opening 326 can all be performed on a multi-axis machine center, such as a 5-axis mill, in a single setup. For example, the jaw pocket 320 may be cut with an end mill approaching from a first direction, which may be the top of the lower jaw 310; the track opening 326 may be cut from the same access direction with a 0.020″ diameter end mill; and the curved needle track 324 may be cut from a side approach using a 0.020″ diameter end mill with a narrow shank extension. The side approach may be from a direction angled in the range of 45° to 135° from the first direction. More specifically, the side approach may be from a direction angled 90° from the first direction. The multi-axis machine may perform cutting, machining, milling, drilling, surfacing, pocketing, boring, facing, parting, or turning operations, among others, to form the track segments or other features of the component. The entry, tangential and exit segments may all be completely exposed to direct line of sight view from outside the jaw component such that there are no obscured undercuts, pockets, or the like, although more than one line of sight may be employed to view all portions of the needle track. Furthermore, the entry, tangential and exit segments may all be accessible to tools, gages, probes, reproduction media, or other instruments along the line(s) of sight. The jaw pocket 320, curved needle track 324 and track opening 326 may all be inspected with direct visual observation from outside the jaw component; there is no need to perform a patency check with a flexible gauge or other secondary inspection methods because patency may be confirmed visually or by direct inspection along one or more lines of sight from outside the jaw component. All features, geometry and/or dimensions of the jaw pocket 320, curved needle track 324 and track opening 326 may be measured directly. It is appreciated that a different size end mill could be used to produce the cuts. It is also appreciated that the jaw pocket 320, curved needle track 324, and track opening 326 could be formed using a wire or plunge EDM process.

The lower jaw 310 disclosed herein may also be manufactured, inclusive of its internal features such as all parts of the needle track, by an injection molding process. A mold body including a selectively shaped mold cavity is prepared. One or more movable cores may be inserted into the mold body to create internal features. A single shot or injection of a bolus of moldable material is injected into the mold body. The core(s) are removed or withdrawn, and the mold body is separated from the molded component. After separation from the mold body, the molded component may be heated to melt out plastic, wax or other materials mixed into the moldable material. The lower jaw and other components described herein may be formed from moldable material comprising one or more of metal, plastic, ceramic, reinforced carbon fiber, or any other moldable material. The lower jaw may also be formed in a single molding process wherein the single molding process is centrifugal casting, die casting, or gravity casting. After the molding operation, the lower jaw 310 may be a single net shape component having substantially all of its final features, geometry, and/or dimensions.

The net shape lower jaw 310 may be left in its as-fabricated condition, or it may undergo subsequent finishing, polishing, plating, passivating, anodizing, coating, etching, reaming, thread chasing, and/or related steps which have minimal or negligible effect on the lower jaw's features, geometry, and/or dimensions. For example, the net shape lower jaw 310 may undergo vibratory finishing, bead blasting, or electropolishing, or it may be sterilized along with the rest of the suture passer.

The jaw link 330 includes a link body 332, and two link holes 332, 334 for connection to the pushrod 264 and the upper jaw 340. The jaw link is contoured to provide clearance with the lower jaw throughout the entire range of motion of the upper jaw. It may be manufactured from stainless steel, and may be machined, or cut with a wire EDM process.

The upper jaw 340 includes an upper jaw body 324 having a first, or distal end 344 and a second, or proximal end 346. The proximal end 346 includes an upper jaw clevis 348 with two sets of holes 350, 352 for linkage to the jaw link 330 and lower jaw 310, respectively. A curved cam surface 354 is formed on the upper jaw proximal end 346. The distal end 344 is forked, and includes a cutout 356 through which the needle guide 314 projects when the jaws are closed. An underside or lower jaw facing side 358 may include serrations, teeth, notches or other features to assist in gripping tissue when the jaws are closed. The upper jaw 340 may also be made by metal injection molding, or casting, and finished on a multi-axis mill with standard milling tools. It may be made so that no surfacing operations are required.

First and second link pins 360 and 362 link the pushrod 264 to the jaw link 330, and link the jaw link 330 to the upper jaw 340. A jaw pin 364 links the upper jaw 340 to the lower jaw 310. These pins may be cut from a standard stainless steel pin size in a trim to length operation.

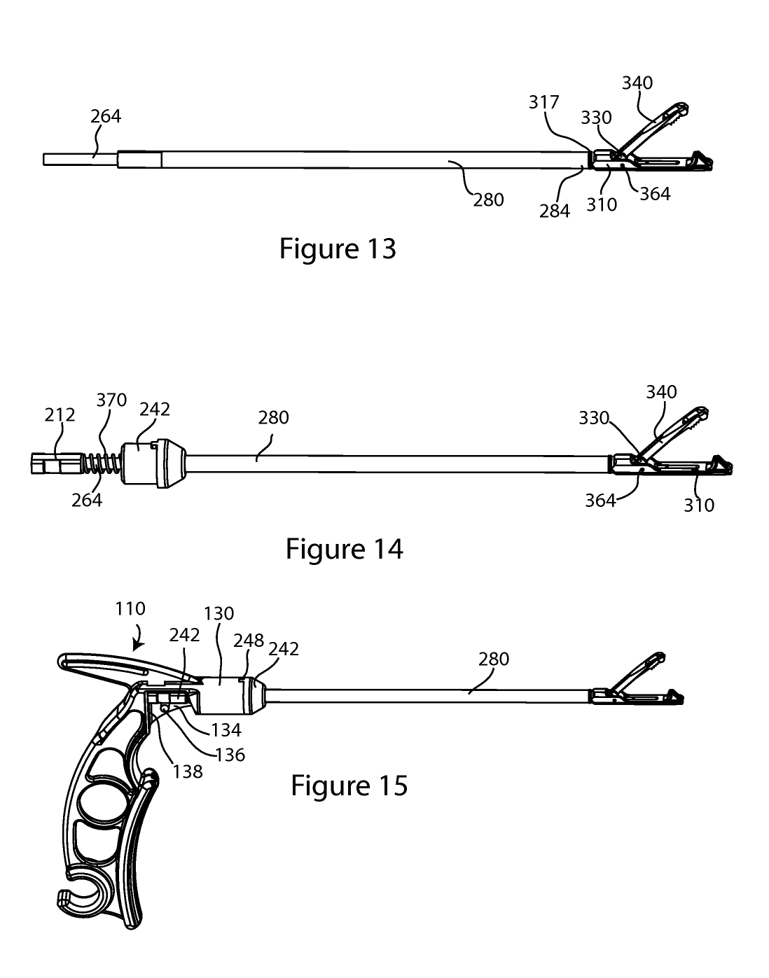

One example of a method of assembly of suture passer 100 is set forth with reference to FIGS. 1, 11 and 13-17. Referring to FIGS. 11 and 13, lower jaw 310 is assembled to the distal end 284 of outer sleeve 280. The lower jaw 310 may be fixed to the outer sleeve 280 using a laser welding process, or a micro TIG (tungsten inert gas) welding process, the flange 317 on the lower jaw 310 providing melt material for the welding.

Referring to FIGS. 9-11 and 14, the pushrod 264 is inserted through the outer sleeve 280 and lower jaw 310 until the pushrod distal end 268 exits the link housing 316. The jaw link 330 is received in the pushrod clevis 274, and the link pin 360 is inserted into holes 276 and 332 to link the jaw link 330 to the pushrod 264. The jaw link 330 is received in the upper jaw clevis 348, and the link pin 362 is inserted into holes 350 and 334 to link the jaw link 330 to the upper jaw 340. The link pins 360, 362 may be welded in place using a laser weld or micro TIG process. The upper jaw 340 is assembled to the lower jaw 310 via jaw pin 364 through holes 318, 352. The jaw pin 364 may be welded in place using a laser weld or micro TIG process. If desired, the outer sleeve 280 may be laser marked, and passivated as necessary.

The outer sleeve proximal end 282 is received in the first bore segment 252 of plug 242. The alignment of the plug on the sleeve is checked, and the plug 242 is glued to the outer sleeve 282. A jaw spring 370 is placed over the proximal end 266 of the pushrod 264, and abuts against the proximal end face 256 of the plug 242. The proximal end 266 of the pushrod 264 is received in the first bore segment 238 of the trigger anvil 212, and rotated relative to the anvil until a proper alignment is reached. The jaw spring 370 is captured between the plug 242 and the trigger anvil 212. The pushrod proximal end 266 is glued to the trigger anvil 212. A gluing fixture may be used to counteract the forces of the spring while the glue cures. Alternatively, the plug 242 may be over-molded to the outer sleeve 280, and the pushrod 264 may be over-molded to the trigger anvil 212.

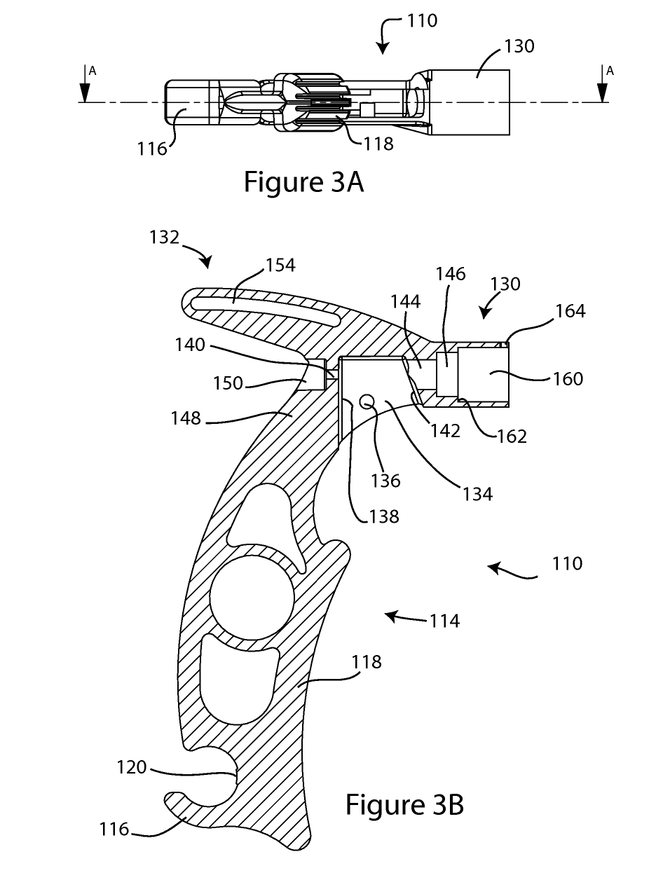

Referring to FIG. 15 and with further reference to FIG. 3B, the assembled trigger anvil 212, pushrod 264, spring 370, and plug 242 are inserted into the barrel portion 130 of the fixed handle 110. The trigger anvil 212, jaw spring 370 and proximal end of the pushrod 264 pass through plug receptacle 160, needle passage 146 and anvil pocket 144, until the anvil second end 232 abuts the proximal wall 138 of the trigger pocket 134. As the anvil second end 232 approaches the trigger pocket 134, the plug body 246 is received in the plug receptacle 160. Proper alignment of the assembly components is ensured by fitting the key 248 into the notch 164. An adhesive may be used to bond the plug body 246 in the receptacle 160.

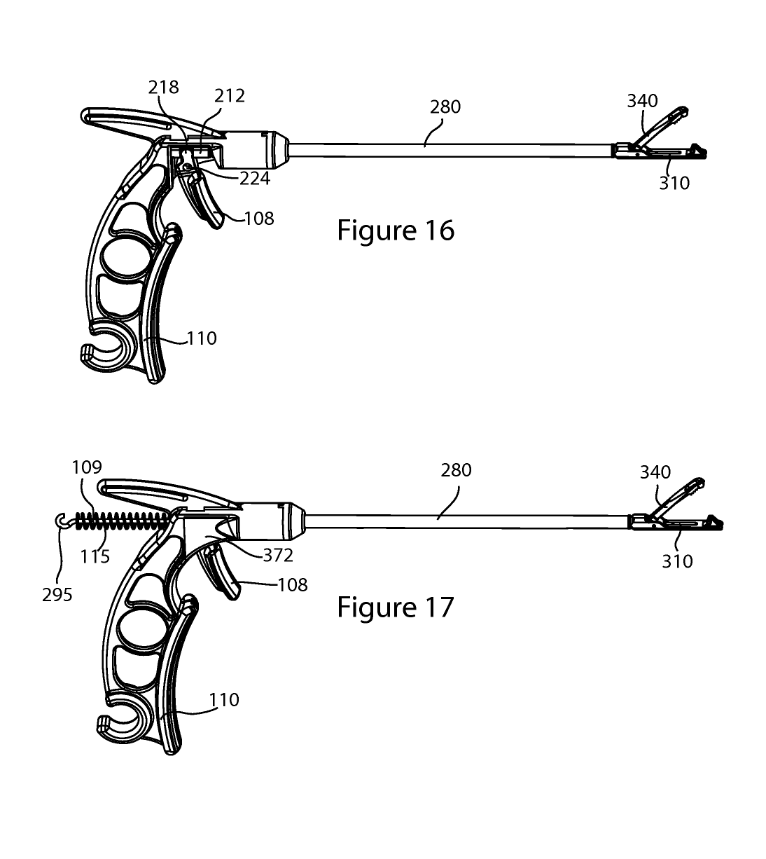

Referring to FIG. 16, the trigger 108 is added to the assembly by fitting the ears 218, 200 about the waist 234 section of the trigger anvil 212, and fitting the trigger bore 224 over the boss 136 of the fixed handle 110. The trigger may be cycled to ensure proper jaw function. When the trigger 108 is depressed or pulled proximally, the force of the trigger actuation overcomes the spring force of jaw spring 370 to pull pushrod 264 proximally and close the jaws 310, 340. When trigger 108 is released, the spring force of jaw spring 370 will push the pushrod 264 distally, opening the jaws 310, 340.

Referring to FIG. 17, main spring 115 is placed onto needle 109, and moved toward the proximal end 294 of the needle until it is seated against the eyelet 295. With reference also to FIGS. 3B, 10, and 12C, the needle 109 is inserted into the fixed handle 110, the needle passing through the first spring pocket 150, the first needle aperture 140, the trigger anvil 212, the anvil pocket 144, the needle passage 146 and into the outer sleeve 280. At the working end 106, the needle 109 passes through the jaw housing 316, across the jaw pocket 320 and into the curved jaw track 324. Optionally an additional pin may be placed in track pin holes 322, crossing over the needle 109. The additional pin may assist in retention of the needle in the needle track, preventing unintended premature exit of the needle through the needle pocket. A handle cap 372 is fitted over the trigger pocket 134 to complete the fixed handle 110, and may provide a portion of the first boss 136.

Referring to FIG. 1, the moving handle 112 is assembled to the fixed handle 110. The range of motion track 154 on the fixed handle 110 is aligned in the clevis 196 of the moving handle 112. The needle eyelet 295, needle shaft 290 and main spring 115 are positioned in the moving handle 112, with eyelet 295 seated vertically in the needle slot 190 and main spring 115 received in spring pocket 188. The ring 180 on the moving handle is snapped into the ring-shaped hinge section 116 on the fixed handle, with tab 120 received in slot 182. Flexing the handles 110, 112 slightly sideways may assist in snapping the ring 180 into the hinge section 116. A first hinge pin 208 is inserted through pin holes 204 and range of motion track 154. A second hinge pin 208 is inserted through pin holes 192 and eyelet 295 to anchor the needle 109 in the moving handle 112.

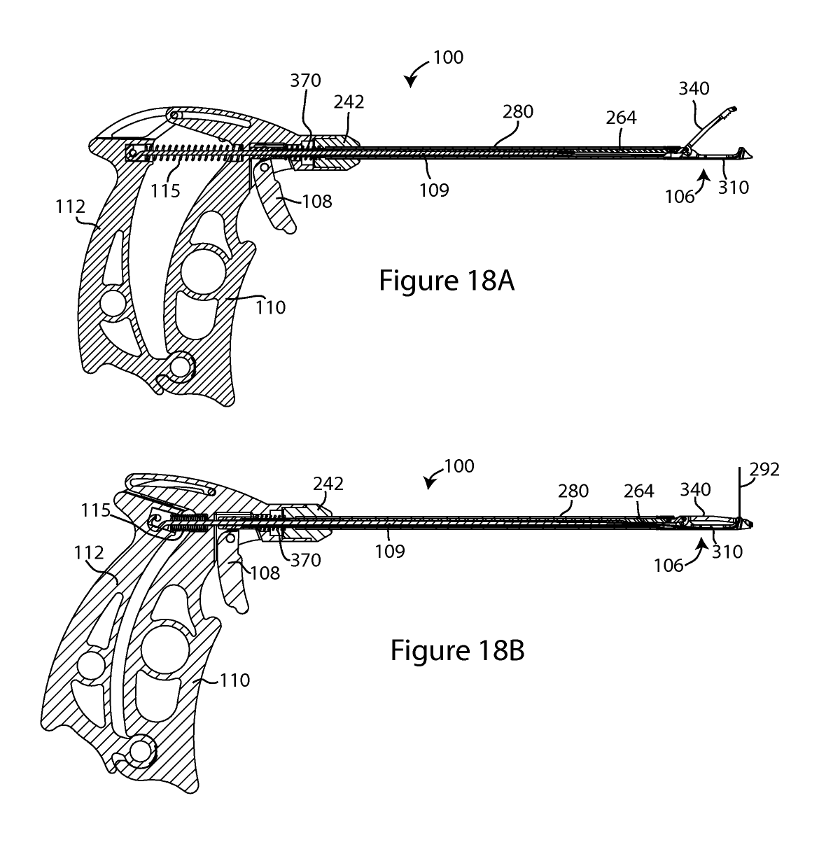

Referring to FIG. 18A, a cross-sectional view shows the suture passer 100 in assembled form. The trigger 108 is in a resting, or non-actuated position, and jaws 310, 340 are open. The spring bias of jaw spring 370 maintains the jaws in the open position until the trigger is actuated to overcome the spring bias. Moving handle 112 is in a first, or resting position, which may be called a proximal position, relative to fixed handle 110. The spring bias of main spring 115 urges the moving handle 112 toward the resting position, and maintains it there until pressure applied to moving handle 112 overcomes the spring bias to move moving handle 112 toward fixed handle 110. The needle 109 is substantially horizontal along its entire length, as it has not been deployed to exit the jaws.

Referring to FIG. 18B, a cross-sectional view shows the suture passer 100 with the trigger 108 actuated, and the needle 109 deployed. Actuation of trigger 108 overcomes the spring bias of jaw spring 370, and pushrod 264 is urged distally to close upper jaw 340 relative to lower jaw 310. Moving handle 112 is in a second, or actuated position, which may which may be called a distal position, relative to fixed handle 110. The pressure on moving handle 112 has overcome the spring bias of main spring 115, and needle 109 is urged distally. A portion of needle ribbon 292 is urged distally through the lower jaw 310 along jaw pocket 320, through curved needle track 324 wherein it is bent tangentially to approximately 90 degrees from its original trajectory, and exits through track opening 326. As the needle passes through the curved track, suture gap 338 and exits the track opening, needle tip 208 may pick up and carry a suture in eye 300, passing the suture through tissue which may be grasped between jaws 310, 340.

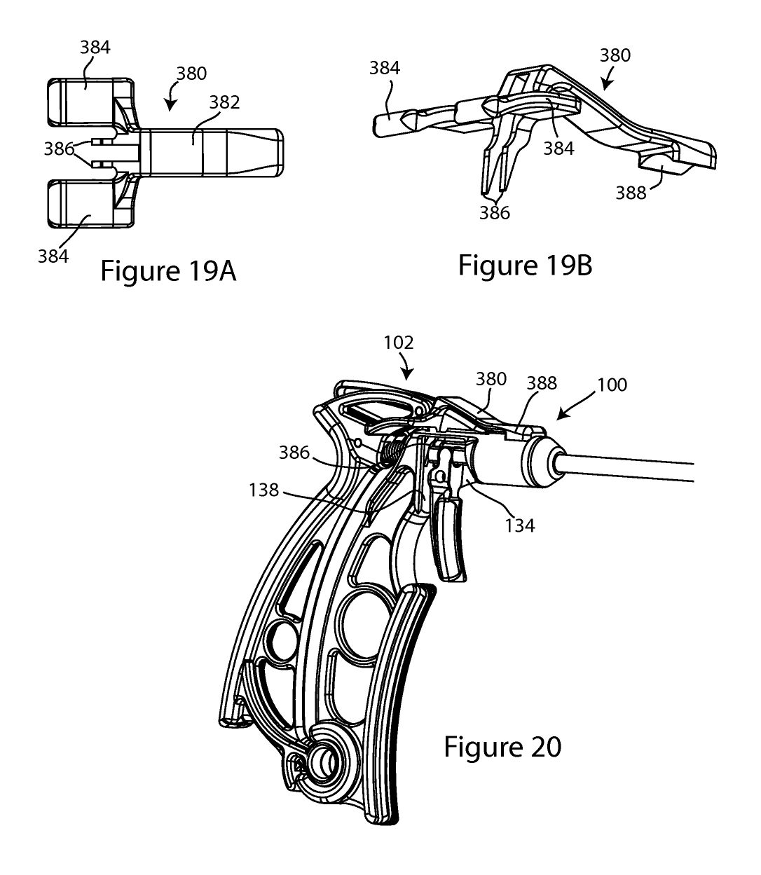

A locking mechanism may be provided to the lock the jaws at any position between and including fully open and fully closed. Referring to FIGS. 19A-19B and 20, a locking member 380 may be deployed to provide a mechanical stop in the form of an obstruction between the trigger anvil 212 and the fixed handle 110. The locking member 380 includes a lock body 382. Projecting from the lock body 382 are a pair of lock tabs 384 and a pair of lock wedges 386. It is appreciated that in other embodiments, the lock member may have only one lock tab and/or lock wedge. A connection portion 388 of the lock body 382 may be permanently or removably attached to the suture passer 100. In the example shown, the lock wedges 386 are positioned to be slidingly inserted between the trigger anvil and the proximal wall 138 of the trigger pocket 134. The wedges 386 are tapered so that any jaw position may be addressed. The locking member 380 is symmetric to the two sides of the handle assembly 102 so as to allow operation in either a right-handed or left-handed configuration. The lock can be applied after the jaw is placed in the desired position by depressing either lock tab 384. In a similar manner, the lock is released by pushing up on either lock tab 384. It is appreciated that in other embodiments, the lock member may be insertable and/or deployable from below, or inferior to the trigger anvil 212. In still other embodiments, a lock member may directly contact the trigger 108 to constrain the trigger in a selected position.

It is appreciated that other methods of creating similar locking actions are contemplated within the scope of the invention. The sliding wedge component does not have to be attached to the trigger, anvil or handle. Alternate locations and motions may include sliding down from above or in from the sides. Advantageously, the locking element may be placed somewhere in the axial components so that the locking force is directly applied to the moving elements and not transmitted through secondary components such as the trigger.

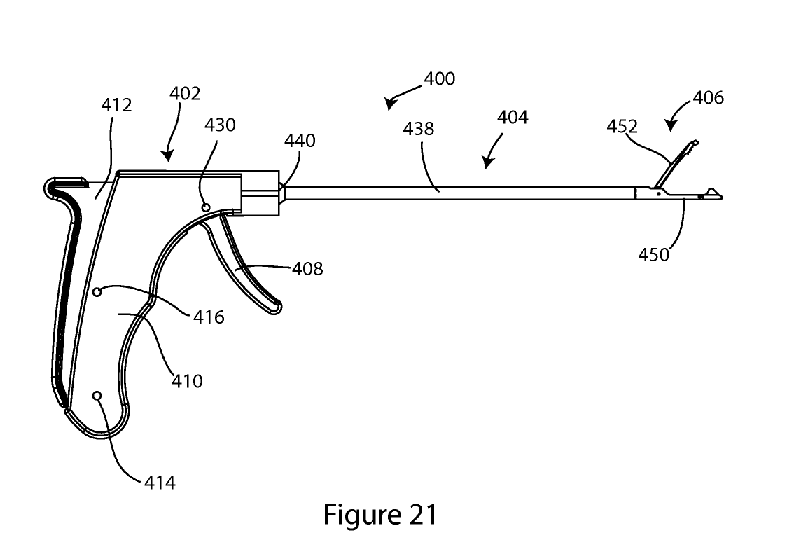

Another example of a disposable and inexpensive to manufacture suture passer is shown in FIGS. 21-24. Suture passer 400 includes a two-piece hinged handle, which may be manufactured through plastic injection molding. The suture passer may comprise a handle assembly 402, a shaft assembly 404, and a working end 406 which may be referred to as a jaw assembly, which includes first and second jaws 450, 452 which may grasp tissue. A needle may be loaded into the handle assembly during assembly. The needle may comprise Nitinol and may have features on the distal tip to engage the suture and subsequently drive it through the tissue. The needle may be urged along a needle track formed in the distal jaws. The needle may bend to conform to a ramped “volcano” at the distal tip and exit 90 degrees from its original axis.

In suture passers known in the art, jaw components can be complicated, expensive parts. To reduce manufacturing complexity and cost, suture passer 400 may include an open needle track with pins placed strategically that act to guide the needle on the appropriate path during its actuation. This may be less expensive to manufacture and use than a suture passer with a closed needle track.

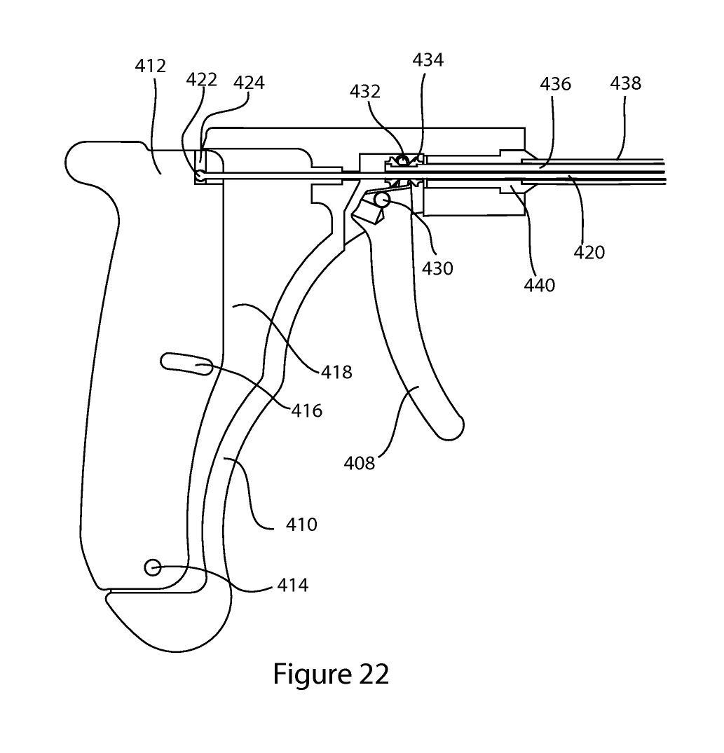

Referring to FIGS. 21 and 22, the handle assembly 402 comprises a movable handle 412 hinged to a rear inset handle 410, which may be a fixed handle. The handles 410, 412 are pivotable relative to one another via a hinge feature 414, which may include a pin or boss received in an opening. A guide feature 416 guides the relative pivoting of the handles, and may include a pin or boss received in a guiding slot. The inset handle 410 includes a recess 418 into which the movable handle 412 can be urged, pivoting about hinge feature 414. A needle 420 is insertable into the handle assembly 402 to pass through the handle, sleeve and rest in an open track of the jaw assembly. The needle includes a retention feature 422, which may be an eyelet, ball or other shape which is retained in a needle slot 424 of the movable handle. Thus as the movable handle 412 is actuated or moved relative to the inset handle 410, the needle 420 is translated distally or proximally between extended and retracted positions. Some or all of the needle may be formed of Nitinol.

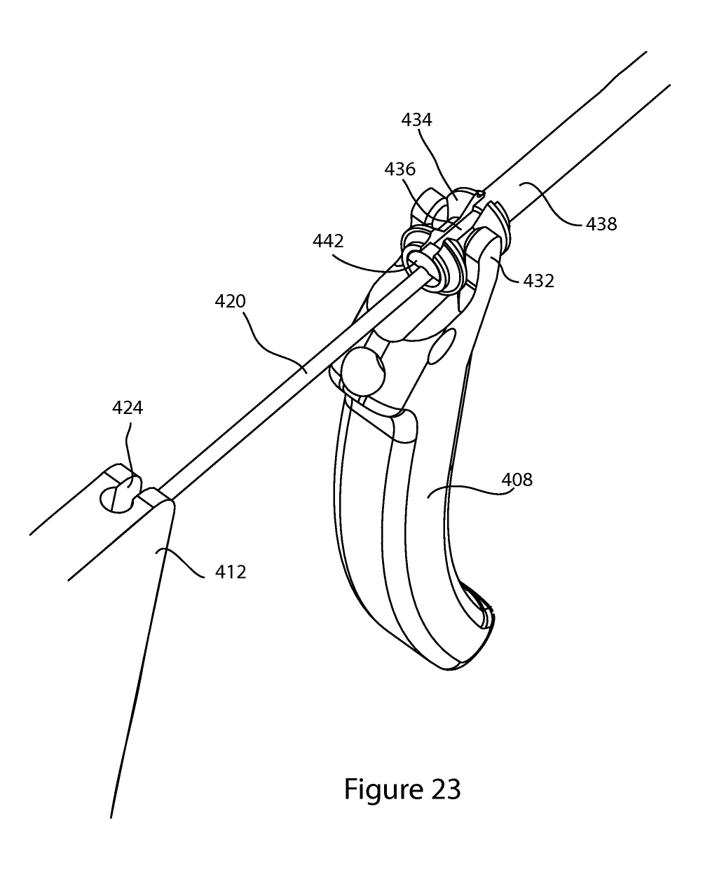

Referring to FIGS. 22 and 23, a trigger 408 extends out of the handle assembly 402. The trigger 408 is pivotable relative to the handle assembly 402 about a trigger boss 430. An engagement tab 432 formed on the trigger 108 engages an actuator 434, which may also be referred to as an anvil. The actuator 434 is connected to a trigger pushrod 436 which extends between the actuator 434 and the second jaw 452. Thus, movement of the trigger 408 about boss 430 engages actuator 434 to translate trigger pushrod 436 to open or close jaws 450, 452. An outer sleeve 438 is connected to a distal end of the handle assembly 402 and extends to the working end 406. A handle plug 440 retains the outer sleeve 438 and provides a passage for the needle 420 and trigger pushrod 436 through the handle 410. A pushrod plug 442 may retain the pushrod 436 proximal end in the actuator 434.

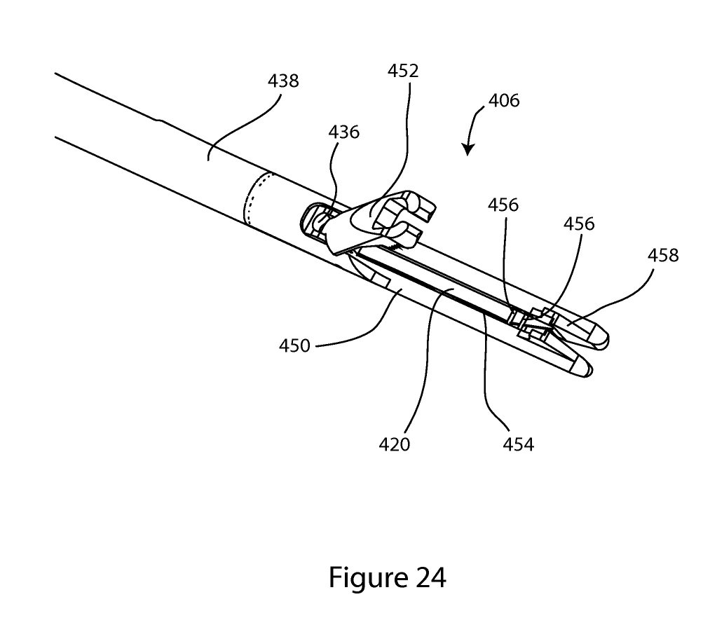

Referring to FIG. 24, the working end 406 or jaw assembly includes the first 450 or lower jaw and the second 452 or upper jaw. A distal end of the trigger pushrod 436 is linked to the second jaw 452 to actuate the jaws. The needle 420 lies in an open needle track 454 formed in the lower jaw 450. One or more pins 456 may extend across the needle track 454 to retain the needle 420 in the track. The more distal guide pin may serve doubly as a guide pin and the main pivot about which the needle will turn or bend. The more proximal guide pin ensures that if the needle tip gets retracted beyond the first pin, it will not be able to spring up and get out of the track. Toward the distal end of first jaw 450, a needle guide 458, which may also be referred to as a volcano, projects superiorly from the jaw. The needle guide 458 includes a ramped portion to guide the needle upward and through tissue which may be clamped between the jaws 450, 452. The natural spring of the Nitinol material may keep the needle against the back surface of the needle track without having a retaining feature near the tip of the volcano. As the needle 420 passes along the needle track 454 and the needle guide 458, it may capture and carry a suture or sutures positioned in the needle guide 458.

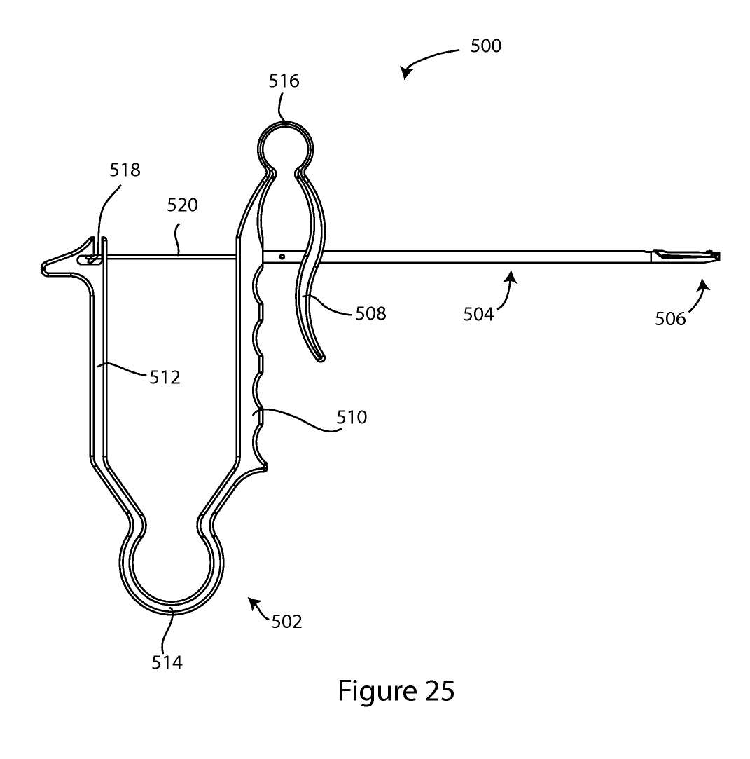

Another example of a disposable and inexpensive to manufacture suture passer is shown in FIG. 25. Suture passer 500 includes a handle assembly 502, a shaft assembly 504, and a working end 506. Working end 506 may have features similar or identical to working ends 106 and/or 406 as previously described. The handle assembly 502 may include a trigger 508, which may be actuable to open and close the jaws of working end 506. The handle assembly 502 may also be actuated to deploy the needle to penetrate and carry the suture through targeted tissue, and retract the needle. These steps may be repeated numerous times.

Handle assembly 502 may include a fixed handle 510, which may be a first handle, and a moving handle 512, which may be a second handle. The fixed handle 510, moving handle 512 and the trigger 508 may consist of a single piece of plastic. Referring to FIG. 25, the moving handle 512 may be situated proximal to the fixed handle 510, and may be connected to the fixed handle 510 via a handle hinge 514, which may be a living hinge, or a two-part hinge similar to the hinges described in previous embodiments. The trigger 508 may be distal to the fixed handle 510 and may be connected to the fixed handle 510 via a trigger hinge 516, which may also be a living hinge, or a two-part hinge. Moving handle 512 may also include a needle-capture feature 518 shaped to capture a proximal end of a needle 520 and to connect the handle assembly 502 to the needle 520.

The needle 520 may extend distally from the needle-capture feature 518 such that it intersects the fixed handle 510 and enters the shaft assembly 504. The shaft assembly 504 may be connected to a distal facing surface of the fixed handle 510 and extend through the trigger 508. The shaft assembly 504 and encapsulated needle 520 may further extend distally to the working end 506.

The material elastic properties of the handle assembly 502 may bias the handles apart from one another and may bias the needle toward a retracted position. A spring (not shown) may also extend between the moving handle 512 and the fixed handle 510 to provide additional bias. The moving handle 512 may translate relative to the fixed handle 510 to actuate the needle such when the handles 510, 512 are squeezed together the operator may overcome the elastic bias and advance the needle into the extended position.

The trigger 508 may be connected to a trigger pushrod (not shown), which may be linked to the upper jaw of the working end 506, similar or the same to that described for suture passer 100. When the trigger 508 is pulled towards the fixed handle 510, the jaws may collapse around the desired tissue. The operator may then apply additional force to overcome the bias between the two handles 510, 512 such that the moving handle 512 is compressed towards the fixed handle 510 and the needle is deployed to pass a suture through the desired tissue. Alternatively, both handles 510, 512 may translate relative to one another when compressed by an operator to overcome the bias and actuate the needle.

Handle assembly 502 may have a length, width and curvature designed to be comfortable in a surgeon's hand. Specifically, the fixed handle 510 may include a plurality of grooves or finger rests shaped to receive the operator's fingers as they grip the handle. Also, the trigger 508 may have an elongated reversed “S”-shape to allow for comfortable grip of the handle 502 for compression. Handle assembly 502 may be formed in a single plastic injection molding process.

The suture passers disclosed herein may be made using methods of manufacture and assembly which present cost and time saving over traditional methods. For one example, suture passer 400 is designed so that the needle 420 travels under pushrod 436 which activates the jaws, as shown in FIG. 23. Pushrod 436 may be manufactured using an injection molding process, and the simplified design of pushrod 436 compared to other pushrods may facilitate an easier injection molding process. To assemble, the pushrod 436 is turned 90 degrees and slipped through the opening of the actuator 434. It can then be rotate back to its starting position and the pushrod plug 442 will then be trapped by the actuator. Once the needle is slid into the assembly, it will prevent the pushrod 436 from rotating in the actuator 434.

For another example, the jaw components of the passers 100, 400, 500 may comprise metal. In some suture passers, such jaw parts can be expensive to machine and may require an intricate laser weld to close off the needle track. In the embodiments depicted, the lower jaw may be made as a single piece instead of a weldment. The needle track is open, instead of closed off and there are a series of ramps and/or pins designed in to guide the needle during initial assembly and keep it in place as the needle is repeatedly deployed. This design may allow the parts to be centrifugally cast more cheaply.

For another example, the needle tracks disclosed herein are open, not closed off. This may present cost saving in at least two aspects. First, a needle track cover does not have to be created and welded or otherwise attached to the jaw to cover the track and prevent unintended escape of the needle from the track. Second, the needle track is open for easy visual inspection, which may save time and costs for inspection personnel and/or equipment.

For yet another example, in suture passers 100 and 400, joints which are typically weld joints may be replaced by glued joints, as glue may provide acceptable strength for a limited use or disposable instrument. This may reduce manufacturing time and/or cost. Joints which may be glued may include the outer sleeve to the plug, and the plug to the fixed handle, among others.

In alternative examples of the suture passers disclosed herein, any of the passers may be tailored to accept multiple suture gages. In another alternative embodiment, the suture passer may be optimized for a specific suture type that the surgeon could choose. Other embodiments may be configured to perform multiple types of stitches, for example, mattress stitches, or multiple sutures. In alternative embodiments, the precise shape of the handle components may vary, for example to provide an ergonomic handle, and/or to provide the most efficient use of materials. Other embodiments may include alternative or additional features on the working end that are compliant to the mechanism of the suture passer, including but not limited to suture cutters, grabbers, clamps, scissors, expanding tips or biopsy instruments.

It should be understood that the present system, kits, apparatuses, and methods are not intended to be limited to the particular forms disclosed. Rather, they are to cover all modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

One way to view the teachings set forth above is to characterize certain methods of manufacture as steps. In the various embodiments set forth above, the step of forming substantially all of the jaw component in a single manufacturing process can be said to be an injection molding process, centrifugal casting, die casting, gravity casting, rapid prototyping, or a multi-axis machining process. The step of confirming patency can be said to be visual confirmation, or direct inspection along one or more lines of sight from outside the component. The step of directly inspecting the geometry of the track can be said to be visual inspection, or inspection with tools, gages, probes, reproduction media, or other instruments along the line(s) of sight from outside the component. The step of introducing a single bolus of material into a mold can be said to be injecting, shooting, pouring, or inserting moldable material into a mold body. The step of mounting a workpiece on a multi-axis machining center can be said to be providing a blank, coupon, workpiece, or mass of raw material and mounting or placing it in the proper position in the machining center for engagement with the tool(s) of the machining center. The step of forming substantially all of the geometry and dimensions of a component on the workpiece without removing the workpiece from the multi-axis machining center can be said to be operating the multi-axis machining center form all of the geometry and dimensions of the component without having to remove and/or re-mount the workpiece in the multi-axis machining center. The step of forming a segment of track in a workpiece can be said to be cutting, machining, milling, drilling, surfacing, pocketing, boring, facing, parting, or turning.

The present invention may be embodied in other specific forms without departing from its spirit or essential characteristics. It is appreciated that various features of the above-described examples can be mixed and matched to form a variety of other alternatives. For example, a trigger assembly from one example may be combined with a jaw and/or needle assembly from another example. Similarly, manufacturing or assembly methods described for one suture passer may be used in manufacture or assembly of another suture passer. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of manufacturing a suture passer, the suture passer having a jaw component having a nonlinear track formed therethrough, the method comprising:
manufacturing the jaw component as a single piece, wherein manufacturing the jaw component consists essentially of forming substantially all of the geometry and dimensions of the jaw component in a single manufacturing process.

2. The method of manufacturing of claim 1, wherein the track enters the jaw component at a first opening and exits the jaw component at an exit opening offset from the first opening.

3. The method of manufacturing of claim 1, wherein the track includes an entry segment, a tangential segment, and an exit segment.

4. The method of manufacturing of claim 3, wherein the tangential segment is in communication with the entry segment and the exit segment, forming a continuous path through the jaw component.

5. The method of manufacturing of claim 3, wherein every segment of the track is directly accessible and directly visible from outside the jaw component.

6. The method of manufacturing of claim 1, the method further comprising:
confirming patency of the track through direct inspection, wherein the entire track is directly visible from outside the jaw component.

7. The method of manufacturing of claim 1, the method further comprising:
directly inspecting the geometry of the track.

8. The method of manufacturing of claim 1, wherein the single manufacturing process is a single molding process.

9. The method of manufacturing of claim 8, wherein the single molding process comprises injecting a single bolus of material.

10. The method of manufacturing of claim 8, further comprising using a single mold body.

11. The method of manufacturing of claim 10, wherein the single molding process further comprises inserting at least one core into the mold body to create an internal feature in the jaw component.

12. The method of manufacturing of claim 8, wherein the jaw component comprises at least one material chosen from the group consisting of: metal, ceramic, plastic, and carbon fiber.

13. The method of manufacturing of claim 8, wherein the single molding process is a process selected from the group consisting of centrifugal casting, die casting, and metal injection molding.

14. The method of manufacturing of claim 1, wherein the single manufacturing process is multi-axis machining.

15. The method of manufacturing of claim 14, the method further comprising:
manufacturing the jaw component using a single setup of a multi-axis machining center.

16. The method of manufacturing of claim 15, wherein manufacturing the jaw component comprises:
mounting a workpiece on the multi-axis machining center; and
forming substantially all of the geometry and dimensions of the jaw component on the workpiece without removing the workpiece from the multi-axis machining center.

17. The method of manufacturing of claim 16, wherein forming substantially all of the geometry and dimensions of the jaw component further comprises:
cutting a first segment of the track into the workpiece from a first direction;
cutting a second segment of the track into the workpiece, the second segment offset from the first segment; and
cutting a third segment of the track into the workpiece from a second direction, the second direction oriented at an angle relative to the first direction.

18. The method of manufacturing of claim 17, wherein the third segment is in communication with the first and second segments to connect the first and second segments to form the track.

19. The method of manufacturing of claim 17, wherein the angle ranges from 45° to 135° relative to the first direction.

20. A method of manufacturing a suture passer, the suture passer having a jaw component having a nonlinear track formed therethrough, the method comprising:
the step of forming substantially all of the jaw component in a single manufacturing process.

21. The method of manufacturing of claim 20, wherein the track enters the jaw component at a first opening and exits the jaw component at an exit opening offset from the first opening.

22. The method of manufacturing of claim 20, wherein the track includes an entry segment, a tangential segment, and an exit segment.

23. The method of manufacturing of claim 22, wherein the tangential segment is in communication with the entry segment and the exit segment, forming a continuous path through the jaw component.

24. The method of manufacturing of claim 22, wherein every segment of the track is directly accessible and directly visible from outside the jaw component.

25. The method of manufacturing of claim 20, the method further comprising:
the step of confirming patency of the track through direct inspection, wherein the entire track is directly visible from outside the jaw component.

26. The method of manufacturing of claim 20, the method further comprising:
the step of directly inspecting the geometry of the track.

27. The method of manufacturing of claim 20, wherein the single manufacturing process is a single molding process.

28. The method of manufacturing of claim 27, wherein the single molding process comprises the step of introducing a single bolus of material into a mold.

29. The method of manufacturing of claim 27, wherein the single molding process further comprises the step of forming the jaw component in a single mold body.

30. The method of manufacturing of claim 29, wherein the single molding process further comprises the step of inserting at least one core into the mold body to create an internal feature in the jaw component.

31. The method of manufacturing of claim 27, wherein the jaw component comprises at least one material chosen from the group consisting of: metal, ceramic, plastic, and carbon fiber.

32. The method of manufacturing of claim 27, wherein the single molding process is a process selected from the group consisting of centrifugal casting, die casting, and metal injection molding.

33. The method of manufacturing of claim 20, wherein the single manufacturing process is multi-axis machining.

34. The method of manufacturing of claim 33, the method further comprising:
the step of manufacturing the jaw component using a single setup of a multi-axis machining center.

35. The method of manufacturing of claim 34, wherein manufacturing the jaw component comprises:
the step of mounting a workpiece on the multi-axis machining center; and
the step of forming substantially all of the geometry and dimensions of the jaw component on the workpiece without removing the workpiece from the multi-axis machining center.

36. The method of manufacturing of claim 35, wherein the step of forming substantially all of the geometry and dimensions of the track further comprises:
the step of forming a first segment of the track in the workpiece from a first direction;
the step of forming a second segment of the track in the workpiece, the second segment offset from the first segment; and
the step of forming a third segment of the track in the workpiece from a second direction, the second direction oriented at an angle relative to the first direction.

37. The method of manufacturing of claim 36, wherein the third segment is in communication with the first and second segments to connect the first and second segments to form the track.

38. The method of manufacturing of claim 36, wherein the angle ranges from 45° to 135° relative to the first direction.

39. A handle assembly for a surgical instrument, the handle assembly comprising:
a first handle and a second handle, the first and second handles pivotably joined by a hinge;
the first handle further comprising an arcuate guide offset from the hinge, the arcuate guide concentric to the hinge;
the second handle further comprising a guided feature;
wherein the arcuate guide cooperates with the guided feature to guide pivotal movement of the second handle relative to the first handle about the hinge.

40. The handle assembly of claim 39, wherein the guided feature, arcuate guide, and hinge are all in the same plane of symmetry.

41. The handle assembly of claim 39, wherein the arcuate guide comprises an arcuate slot, wherein the guided feature comprises a pin, wherein the pin travels in the arcuate slot to constrain the pivotal movement of the second handle relative to the first handle.

42. The handle assembly of claim 41, wherein the guided feature further comprises a clevis formed on the second handle, wherein the clevis carries the pin.

43. The handle assembly of claim 39, further comprising a spring captured between the first handle and the second handle, the spring biasing the handle assembly toward an open configuration.

44. The handle assembly of claim 43, wherein the spring lies between the hinge and the arcuate guide.

45. The handle assembly of claim 39, further comprising an instrument shaft having a shaft axis, wherein a proximal end of the instrument shaft is seated in the handle assembly, wherein the pivotal movement of the second handle relative to the first handle translates the instrument shaft along the shaft axis.

46. The handle assembly of claim 45, wherein the second handle further comprises a shaft seat, wherein the proximal end of the instrument shaft is seated in the shaft seat.

47. The handle assembly of claim 45, wherein the instrument shaft lies between the hinge and the arcuate guide.

48. The handle assembly of claim 45, wherein the shaft axis, hinge, guide feature and arcuate guide all lie in the same plane of symmetry.

49. The handle assembly of claim 39, wherein the hinge comprises a first ring and a second ring, the first and second rings fitted concentrically together, the first and second rings pivotably movable relative to one another.

50. The handle assembly of claim 49, wherein the first handle comprises the first ring and the second handle comprises the second ring.

51. The handle assembly of claim 49, wherein the first ring comprises a tab and the second ring comprises a slot, wherein the tab is received in the slot to lock the first and second rings concentrically together.

52. The handle assembly of claim 39, wherein the first and second handles are formed by an injection molding process.
